(19) United States
(12) Patent Application Publication
(10) Pub. No.: US 2005/0281754 A1
(43) Pub. Date: Dec. 22, 2005

(54) Sprayable compositions comprising a combination of pharmaceutical active ingredients, an alcohol phase and an oily phase

(75) Inventors: Nathalie Willcox, Saint Vallier De Thiey (FR); Sandrine Orsoni, Mandelieu (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A., CHAM (CH)

(21) Appl. No.: 10/951,903

(22) Filed: Sep. 29, 2004

(30) Foreign Application Priority Data
Jun. 17, 2004 (FR) 04/06616

(51) Int. Cl. A61K 9/00; A61K 31/59

(52) U.S. Cl. 424/47; 514/167; 514/171

(57) ABSTRACT

Sprayable, anhydrous and physically/chemically stable dermatological/pharmaceutical compositions, well suited for the treatment of a variety of dermatological disorders, notably psoriasis, contain: a) a therapeutically effective amount of a solubilized corticoid, notably dissolved clobetasol propionate; b) a therapeutically effective amount of a solubilized vitamin D derivative, notably dissolved calcitriol; and c) an alcohol phase; and d) an oily phase which comprises one or more oils; formulated into e), a sprayable and topically applicable, dermatologically/pharmaceutically acceptable vehicle therefor.

### CROSS-REFERENCE TO PRIORITY APPLICATION

This application claims priority under 35 U.S.C. § 119 of FR 04/06616, filed Jun. 17, 2004, hereby expressly incorporated by reference and assigned to the assignee hereof.

### BACKGROUND OF THE INVENTION

1. Technical Field of the Invention

The present invention relates to anhydrous compositions in the form of a spray comprising a combination of clobetasol propionate (corticord) and calcitriol (vitamin D derivative) as pharmaceutical active ingredients, an alcohol phase and an oily phase in a physiologically acceptable medium, to the process for the preparation of same and to cosmetic and dermatology applications thereof.

2. Description of Background and/or Related and/or Prior Art

It is not conventional to use a combination of active principles in the treatment of dermatological complaints. The main difficulties encountered by one skilled in the art when combining two active principles are the problems of chemical instability and the interactions which the active principles may initiate when they are present in the same formulation.

Few treatments therefore exist which combine calcitriol and a corticoid. In fact, vitamin D and its derivatives are unstable in aqueous media and sensitive to acidic pH values, whereas corticoids, and more particularly clobetasol propionate, are sensitive to basic media. It was not therefore obvious to one skilled in the art to combine and stabilize an active ingredient of the vitamin D type and a corticosteroid in one and the same composition.

Calcitriol is a vitamin D analogue used to regulate the calcium level in the organism. Its use in the treatment of dermatological diseases has been described especially in U.S. Pat. No. 4,610,978 for the treatment of psoriasis. Said patent suggests compositions comprising calcitriol that can also contain an amount of an anti-inflammatory such as a corticosteroid, but no concrete embodiment of a combination of calcitriol and a corticosteroid is either described or tested in terms of efficacy.

FR-2,848,454, assigned to the assignee hereof, describes that a combination of calcitriol with a corticosteroid made it possible to obtain a synergistic effect in the treatment of certain dermatological complaints such as psoriasis, atopic dermatitis, contact dermatitis and seborrhoeic dermatitis, without however proposing stable pharmaceutical compositions combining both active ingredients.

Furthermore, in the field of dermatology and the formulation of pharmaceutical compositions, one skilled in the art seeks compositions which not only have to be physically and chemically stable, but also have to make it possible to release the active ingredient and promote its penetration through the cutaneous layers so as to improve its efficacy.

The pharmaceutical compositions moreover have to have a good cosmetic character and preferably be non-irritant.

There are currently numerous topical compositions that comprise an active ingredient and are capable of promoting its penetration into the skin by virtue of the presence especially of a high content of propenetrating glycol. These compositions are formulated as emulsions with a high content of fatty phase, commonly called “lipocreams”, as anhydrous compositions called “unguents”, as fluid compositions with a high content of volatile solvents such as ethanol or isopropanol, intended for application to the scalp and also called “hair lotions”, or as viscous O/W emulsions, also called “O/W creams”.

The stabilization of a formulation comprising such a percentage of glycol makes it necessary to use, in the emulsion, emulsifiers and stabilizers of the glyceryl stearate or PEG 100 stearate type, or stabilizers or consistency factors of the white wax or cetostearyl alcohol type, which give rise to the formation of a viscous cream, i.e., a cream with a viscosity greater than 10 Pa.s (10,000 centipoises, measured with a Brookfield LVDV II apparatus+no. 4 cup, at a speed of 30 rpm for 30 seconds and at a temperature of 25° C.±3° C.). This viscosity therefore makes the product difficult to apply. Hence, these compositions, on the one hand, have a poor cosmetic acceptability due to their viscosity, and, on the other hand, carry risks of intolerance caused by the presence of high proportions of glycol. In addition, these high viscosities make the formulations difficult to apply to the different parts of the body affected by the pathological condition. Consequently, the majority of existing treatments, in the form of creams, gels or ointments, require the help of a third party to apply them to the areas that are difficult to reach. The third party therefore has to touch both the product containing the active ingredient and the psoriatic plaques, resulting in a situation that is not ideal from the point of view of the comfort of the user and the safety of the third party. One skilled in the art is also aware that non-compliance with the prescribed treatment for reasons referred to above is one of the main causes of failure, the article “Patients with psoriasis and their compliance with medication” (Richards et al., J. Am. Acad. Dermatol., Oct. 99, pp 581-583) indicating that nearly 40% of patients with a chronic disease like psoriasis do not follow their treatment. It has been demonstrated that the patient's compliance with his treatment is directly related to the characteristics of the vehicle of the composition applied. The article “Patients with psoriasis prefer solution and foam vehicles: a quantitative assessment of vehicle preference” (Housman et al., CUTIS, Dec. 2002, vol. 70, pp 327 to 332) indicates that psoriasis patients prefer a solution or a foam to an unguent, a cream or a gel.

It thus appears desirable to improve the comfort on use of this type of composition, which is what is accomplished by the present invention described hereinbelow.

The prior art closest to the invention is WO 00/64450, which indicates the use of a pharmaceutical composition containing a vitamin D analogue and a corticosteroid. All the composition examples in said patent application combine solely calcipotriol and betamethasone dipropionate. The preferred compositions described in the patent application that make it possible to stabilize the two active ingredients are compositions in the form of an unguent. However, these compositions exhibit the abovementioned disadvantages as regards comfort and ease of application. Study of this prior art in no event suggests to those skilled in the art sprayable, i.e., easily applicable, compositions such as those described herein with the active ingredients clobetasol propionate and calcitriol, which are solubilized and stable in the composition.

### SUMMARY OF THE INVENTION

The problem which the present invention solves is the provision of a physically and chemically stable composition that allows the two active ingredients calcitriol and clobetasol propionate to be combined in one and the same composition, said ingredients acting synergistically for the treatment of psoriasis, and the compositions according to the invention also being easy to use and having an acceptable cosmetic character for application to all areas of the body that may be affected by the pathological condition.

“Physical stability” is understood according to the invention as applying to a composition that does not undergo any modification of macroscopic appearance (phase separation, change of color or appearance, etc.) or microscopic appearance (recrystallization of active ingredients) after storage at temperatures of 4° C. and 40° C. for 2, 4, 8 and 12 weeks.

“Chemical stability” is understood according to the invention as applying to a composition in which the active principle content remains stable after three months at room temperature and at 40° C. A stable active principle content means according to the invention that the content varies very little relative to the initial content, i.e., that the variation in active principle content at time T must not be less than 90% of the initial content at T0 and preferably not less than 95% of the initial content at T0.

Thus, it has now surprisingly been found that compositions comprising, formulated into a pharmaceutically acceptable vehicle therefor:

a) a therapeutically effective amount of a corticoid in solubilized form, and more particularly clobetasol propionate (or clobetasol 17-propionate);

b) a therapeutically effective amount of a vitamin D derivative in solubilized form, and more particularly calcitriol;

c) an alcohol phase; and

d) an oily phase which comprises one or more oils,

and being in the form of a spray, constitute compositions which ameliorate or avoid the above disadvantages and drawbacks of the prior art.

While allowing a good penetration of the active principles, the compositions of the present invention are chemically and physically stable. They also have a very good patient acceptability and tolerance, due to their spray formula, as described below in the examples of the present invention. The compositions according to the invention are therefore particularly suitable for the treatment of dermatological complaints, conditions and afflictions and more particularly for the treatment of psoriasis.

The present invention therefore features sprayable compositions comprising the following, in a pharmaceutically acceptable vehicle:

a) a therapeutically effective amount of clobetasol propionate in solubilized form;

b) a therapeutically effective amount of calcitriol in solubilized form;

c) an alcohol phase; and

d) an oily phase which comprises one or more oils.

### DETAILED DESCRIPTION OF BEST MODE AND SPECIFIC/PREFERRED EMBODIMENTS OF THE INVENTION

Advantageously, the compositions according to the invention comprise from 0.00001 to 0.1% by weight, preferably from 0.0001 to 0.001% by weight and particularly preferably from 0.0002 to 0.0005% by weight of an active ingredient derived from vitamin D, based on the total weight of the composition. The compositions according to the invention comprise more particularly 0.0003% by weight of calcitriol, based on the total weight of the composition.

Advantageously, the compositions according to the invention comprise from 0.0001 to 0.1% by weight and preferably from 0.001 to 0.05% by weight of a corticoid, based on the total weight of the composition. The preferred compositions according to the invention comprise more particularly 0.025% or 0.05% by weight of clobetasol propionate, based on the total weight of the composition.

“Alcohol phase” is understood according to the invention as meaning at least one alcohol compound. Non-limiting examples which may be mentioned of alcoholic compounds usable according to the invention are linear or branched aliphatic alcohols such as anhydrous ethanol, isopropanol and butanol. The compositions according to the invention preferably contain ethanol. Advantageously, the compositions contain from 30 to 60% by weight and preferably from 45 to 55% by weight of an alcohol, based on the total weight of the composition.

A preferred composition according to the invention contains from 45 to 55% by weight of ethanol.

“Oily phase” is understood according to the invention as meaning an oily phase that is appropriate for a pharmaceutical or cosmetic composition. Oils generally have a viscosity above about 10 centipoises at 25° C. and can reach a viscosity ranging up to 1,000,000 centipoises at 25° C. The oil can be one of a wide variety of synthetic or natural silicone or organic oils, a non-exhaustive list of which is given by way of indication.

(a) Esters:

Examples of oils usable according to the invention comprise esters of the formula RCO—OR′, where R and R′, which are identical or different, are a linear or branched alkyl, alkenyl, alkoxycarbonylalkyl or alkoxycarbonyloxyalkyl chain having from 1 to 25 carbon atoms and preferably from 4 to 20 carbon atoms. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coconut dicaprylate/caprate, decyl isostearate, isodecyl oleate, isodecyl neopentanoate, isohexyl neopentanoate, octyl palmitate, dioctyl malate, tridecyl octanoate, myristyl myristate and octyldodecanol.

(b) Fatty acid glyceryl esters:

The oil can also comprise fatty esters of natural fatty acids, or triglycerides of animal or vegetable origin. Examples of these include castor oil, lanolin oil, triisocetyl citrate, triglycerides having from 10 to 18 carbon atoms, caprylic/capric triglycerides, coconut oil, maize oil, cottonseed oil, linseed oil, mink oil, olive oil, palm oil, mahua butter, colza oil, soya oil, sunflower oil, walnut oil, sweet almond oil, wheatgerm oil, jojoba oil and equivalent compounds.

(c) Fatty acid glycerides:

Other suitable oils are synthetic or semisynthetic glyceryl esters such as fatty acid mono-, di- and triglycerides, which are modified natural oils or fats, for example glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates and equivalent compounds.

(d) Non-volatile hydrocarbons:

Other very suitable solvents for the composition according to the invention are non-volatile hydrocarbons such as paraffins, isoparaffins, mineral oils and equivalent compounds.

(e) Guerbet esters:

Guerbet esters are esters resulting from the reaction of a Guerbet alcohol of the general formula: 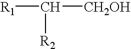
with a carboxylic acid of the general formula:
R³COOH or HOOC—R₃—COOH,
in which R₁ and R₂, which are identical or different, are an alkyl having from 4 to 20 carbon atoms and R₃ is a substituted or unsubstituted fatty radical such as a saturated or unsaturated, linear or branched alkyl or alkylene chain having from 1 to 50 carbon atoms, or a phenyl capable of being substituted by a halogen, a hydroxyl, a carboxyl or an alkylcarbonylhydroxyl.

The Guerbet alcohols mentioned above, especially those of the octyidodecanol type marketed under the name Eutanol G, are also suitable for the composition according to the invention.

Mention may also be made of volatile silicone oils, such as linear siloxanes and more preferably hexamethyldisiloxane. By way of example, mention may be made of the product DC Fluid 0.65cSt marketed by Dow Corning.

Preferably, the oily phase of the composition according to the invention comprises one or more oils selected from among the caprylic/capric triglycerides marketed under the name Miglyol 812, the cetearyl isononanoate marketed under the name Cetiol SN, and vegetable oils (sweet-almond oil, sesame oil, wheatgerm oil, olive oil, jojoba oil, etc.).

Advantageously, the compositions according to the invention comprise from 5 to 80% by weight, preferably from 20 to 70% by weight and particularly preferably from 40 to 60% by weight of oily phase, based on the total weight.

The compositions according to the invention thus comprise, in a pharmaceutically acceptable vehicle:

a) from 0.0001 to 0.1% of clobetasol propionate;

b) from 0.00001 to 0.1% of calcitriol;

c) from 30 to 60% of ethanol;

d) from 5 to 80% of an oily phase which comprises one or more oils selected from among caprylic/capric triglycerides, cetearyl isononanoate and vegetable oils.

In one preferred embodiment, the compositions according to the invention also contain antioxidant compounds such as DL-α-tocopherol, butylhydroxyanisole or butylhydroxytoluene, propyl gallate, superoxide dismutase, ubiquinol or certain metal chelating agents. The antioxidants preferably included in the compositions according to the invention are DL-α-tocopherol, butylhydroxyanisole and butylhydroxytoluene.

The compositions according to the invention can also contain surfactants. The surfactants usable according to the invention are of the anionic surfactant type such as carboxylates and especially soaps, alkylarylsulfonates, alkylethersulfates, alkylsulfates and alcohol sulfates. More particularly, the anions of these surfactants are coupled with a cation such as that of the metal sodium or potassium. Other preferred surfactants according to the invention are those of the polysorbate and poloxamer types.

Preferably, the surfactants used according to the present invention are sodium laurylsulfate, polysorbate 80 (TWEEN 80 from Uniqema) and poloxamer 124 (SYNPERONIC PEL44 from Uniqema).

The pharmaceutical compositions according to the invention may also contain inert additives or combinations of these additives, such as

wetting agents;

flavor improvers;

preservatives;

stabilizers;

humidity regulators;

pH regulators;

osmotic pressure modifiers;

emulsifiers;

UV-A and UV-B filters;

propenetrating agents; and

synthetic polymers.

Of course, those skilled in the art will take care to choose any compound(s) to be added to these compositions in such a way that the advantageous properties intrinsically associated with the present invention are unaffected or substantially unaffected by the envisaged addition.

The compositions according to the invention are more particularly suited for a regime or regimen for the treatment of the skin and the mucosae; they are sprayable and suitable for packaging in the form of a spray.

The spray has numerous advantages compared with conventional forms, such as easy delivery of the formula to the areas of the body which are very difficult to treat, possible simple control of the dose delivered or the absence of contamination during use.

The compositions according to the invention are therefore administered in the form of a sprayable composition. The latter can be obtained by conventional formulating means known to those skilled in the art. For example, the compositions can be sprayed by a mechanical sprayer which pumps the composition from a container, bottle or equivalent vessel. Likewise, the compositions can be propelled by means of a gas in the manner well known to those skilled in the art. The conventional propellant gases, such as air or hydrocarbons, are effective provided they do not interfere with the composition. The composition passes through a nozzle, which can be pointed directly at the desired application site. The nozzle can be chosen so as to apply the composition in the form of a vapor or a jet of droplets according to the techniques known to those skilled in the art. Depending on the chosen pharmaceutical active ingredient, the spraying mechanism must be capable always of dispensing the same amount of active ingredient. The mechanisms for controlling the amount of composition to be dispensed by the spray are also known to those skilled in the art. For example, the amount of propellant gas can be calculated so as to propel the exact amount of product desired. For the compositions according to the invention, it is possible to use a dosing vaporizer bottle whose characteristics of application area and dose are controlled and reproducible. For example, the vaporizer can consist of a bottle equipped with a dosing valve.

While allowing a good penetration of the active principles, the compositions of the present invention are chemically and physically stable. They also have a very good patient acceptability and tolerance, due to their spray formula, as described in the examples of the present invention. The compositions according to the invention are therefore found to be particularly suitable for the treatment of dermatological complaints or disorders.

The present invention therefore also features the formulation of a composition according to the invention for the preparation of a drug suited for the treatment of:

dermatological complaints or disorders associated with a keratinization disorder related to differentiation and proliferation, especially acne vulgaris, black heads, polymorphous acne, acne rosacea, nodulocystic acne, acne conglobata, senile acne, and secondary acne such as solar acne, acne medicamentosa or occupational acne;

ichthyosis, ichthyosiform states, Darrier's disease, palmoplantar keratoderma, leukoplakia and leukoplakiform states, and cutaneous or mucous (buccal) lichen;

dermatological complaints or disorders having an inflammatory immunoallergic component and with or without cellular proliferation disorder, especially cutaneous, mucous or ungueal psoriasis, psoriatic rheumatism, and cutaneous atopy such as eczema, respiratory atopy or gingival hypertrophy;

benign or malignant dermal or epidermal proliferations of viral or non-viral origin, especially verrucas, plane warts, epidermodysplasia verruciformis, oral or florid papillomatosis, and T lymphoma;

proliferations inducible by ultraviolet, especially basal cell and spinal cell epithelioma;

precancerous cutaneous lesions, especially keratoacanthomas;

immune dermatoses, especially lupus erythematosus;

bullous immune diseases;

collagen diseases, especially scleroderma;

dermatological or systemic complaints or disorders having an immunological component;

cutaneous disorders due to exposure to UV radiation, photoinduced or chronological aging of the skin, or actinic pigmentations and keratoses, or any pathological conditions associated with chronological or actinic aging, especially xerosis;

sebaceous function disorders, especially hyperseborrhoeic acne, simple seborrhoea or seborrhoeic dermatitis;

healing or cicatrization disorders or striae atrophicae;

pigmentation disorders such as hyperpigmentation, melasma, hypopigmentation or vitiligo;

disorders of lipid metabolism, such as obesity, hyperlipidaemia, non-insulin-dependent diabetes or syndrome X;

inflammatory complaints or disorders such as arthritis;

cancerous or precancerous states;

alopecia of different origins, especially that due to chemotherapy or radiation;

immune system disorders such as asthma, type I sugar diabetes, multiple sclerosis or other selective dysfunctions of the immune system; or

disorders of the cardiovascular system, such as arteriosclerosis or hypertension.

In a preferred embodiment according to the invention, the subject compositions are used for the preparation of a drug suitable for treating psoriasis.

In particular, the compositions as defined above comprise 0.0025% of clobetasol 17-propionate and 0.0003% of calcitriol in the presence of ethanol.

The examples which follow are a non-exhaustive representation of formulation examples of the composition according to the invention, together with chemical and physical stability results and results of the test of release-penetration of the active ingredients.

In said examples to follow, all parts and percentages are given by weight, unless otherwise indicated.

### EXAMPLE 1

### Stability of Calcitriol in Various Excipients

The following example describes the calcitriol stability data in various excipients, including ethanol 100, caprylic/capric triglycerides and cetearyl isononanoate, preferred excipients for the compositions according to the invention.

a) Stability of calcitriol in ethanol:

Solution of 30 ppm of calcitriol in qsp 100% of absolute ethanol, in the presence of 0.02% of BHT.

Technique of HPLC assay against a reference substance.

At the starting time (T0) the composition is considered to comprise 100% of calcitriol. 5

Measured concentration of calcitriol in % relative to T0:

| Stability | T 1 | T 2 | T 3 | T 4 |
|---|---|---|---|---|
| conditions | week | weeks | Weeks | weeks |
| −18° C. | 100.9% | 100.5% | 99.5% | 99.5% |
| +4° C. | 97.7% | 98.6% | 98.1% | 97.7% |
| +30° C. | / | 93.4% | / | 93.0% |

b) Stability of calcitriol in Miglyol 812 (caprylic/capric triglycerides):

Solution of 30 ppm of calcitriol in qsp 100% of Miglyol 812, in the presence of 0.4% of BHT.

Technique of HPLC assay against a reference substance.

At the starting time (T0) the composition is considered to comprise 100% of calcitriol.

Measured concentration of calcitriol in % relative to T0:

| Stability | | |
|---|---|---|
| conditions | T 2 weeks | T 4 weeks |
| +4° C. | 98.3% | 105.2% |
| RT | 95.1% | 98.0% |
| +40° C. | 91% | 93.0% |

c) Stability of calcitriol in Cetiol SN (cetearyl isononanoate):

Solution of 30 ppm of calcitriol in qsp 100% of Cetiol SN (cetearyl isononanoate), in the presence of 0.4% of BHT

Technique of HPLC assay against a reference substance.

At the starting time (T0) the composition is considered to comprise 100% of calcitriol.

Measured concentration of calcitriol in % relative to T0:

| Stability | | |
|---|---|---|
| conditions | T 2 weeks | T 4 weeks |
| +4° C. | 98.6% | 98.1% |
| RT | 98.7% | 98.4% |
| +40° C. | 99.0% | 98.9% |

### EXAMPLE 2

### Process for the Preparation of the Compositions According to the Invention

The compositions according to the invention are prepared at room temperature, under a hood and in inactinic light.

The antioxidant, the calcitriol and the alcohol are introduced into a flask and stirred until the calcitriol is perfectly solubilized.

The clobetasol propionate is then added and stirring is continued until the clobetasol propionate is solubilized.

When the two active ingredients are perfectly solubilized, the remaining constituents of the formulation are introduced in succession.

The mixture is stirred until it is perfectly homogeneous.

### EXAMPLE 3

| CONSTITUENTS | % |
|---|---|
| 2-PROPANOL | qs 100 |
| DL-ALPHA-TOCOPHEROL ACETATE | 0.04 |
| CALCITRIOL | 0.0003 |
| CLOBETASOL 17-PROPIONATE | 0.001 |
| SESAME OIL | 5 |
| MEDIUM CHAIN TRIGLYCERIDES | 55 |
| POLOXAMER 124 | 0.10 |

The procedure is the one described in Example 2.

A slightly yellow liquid solution is obtained.

### EXAMPLE 4

| CONSTITUENTS | % |
|---|---|
| ABSOLUTE ETHANOL | qs 100 |
| BUTYLHYDROXYTOLUENE | 0.04 |
| CALCITRIOL | 0.0003 |
| CLOBETASOL 17-PROPIONATE | 0.025 |
| ALMOND OIL | 5 |
| MEDIUM CHAIN TRIGLYCERIDES | 55 |
| POLYSORBATE 80 | 0.10 |

The procedure is the one described in Example 2.

A slightly yellow liquid solution is obtained.

### EXAMPLE 5

| CONSTITUENTS | % |
|---|---|
| ABSOLUTE ETHANOL | qs 100 |
| BUTYLHYDROXYTOLUENE | 0.04 |
| CALCITRIOL | 0.0003 |
| CLOBETASOL 17-PROPIONATE | 0.025 |
| 1,2-PROPANEDIOL | 10 |
| MEDIUM CHAIN TRIGLYCERIDES | 35 |
| ALMOND OIL | 5 |
| POLYSORBATE 80 | 0.10 |

The procedure is the one described in Example 2.

A slightly yellow liquid solution is obtained.

### EXAMPLE 6

| CONSTITUENTS | % |
|---|---|
| ABSOLUTE ETHANOL | qs 100 |
| BUTYLHYDROXYTOLUENE | 0.04 |
| CALCITRIOL | 0.0003 |
| CLOBETASOL 17-PROPIONATE | 0.025 |
| 1,2-PROPANEDIOL | 10 |
| MEDIUM CHAIN TRIGLYCERIDES | 40 |
| POLYSORBATE 80 | 0.10 |

The procedure is the one described in Example 2.

A colorless liquid solution is obtained.

### EXAMPLE 7

| CONSTITUENTS | % |
|---|---|
| ABSOLUTE ETHANOL | qs 100 |
| BUTYLHYDROXYTOLUENE | 0.04 |
| CALCITRIOL | 0.0003 |
| CLOBETASOL 17-PROPIONATE | 0.025 |
| MEDIUM CHAIN TRIGLYCERIDES | 40 |
| POLYSORBATE 80 | 0.10 |

The procedure is the one described in Example 2.

The composition obtained is a clear liquid solution.

### EXAMPLE 8

### Physical Stability of the Composition According to Example 6

The physical stability of the formulations is measured by macroscopic and microscopic observation of the formulation at room temperature, at 4° C. and at 40° C. after 2, 4, 8 and 12 weeks.

At room temperature, macroscopic observation makes it possible to guarantee the physical integrity of the products and microscopic observation makes it possible to verify that there is no recrystallization of the solubilized active ingredient.

Non-recrystallization of the solubilized active ingredients is verified by microscopic observation at 4° C.

The integrity of the finished product is verified by macroscopic observation at 40° C.

Specifications at T0:

Macroscopic appearance: colorless liquid spray

Microscopic appearance: absence of crystals of calcitriol and clobetasol 17-propionate

| | Time |
|---|---|
| Stability conditions | T 15 days |
| RT | conforms to the specification |
| +4° C. | conforms to the specification |
| +40° C. | conforms to the specification |

### EXAMPLE 9

### Chemical Stability of the Active Ingredients Within the Composition According to Example 6

Stability of the calcitriol:

Assay of the active ingredient is carried out by external calibration using HPLC.

The results are expressed in % recovery relative to the theoretical value.

| | Time |
|---|---|
| Stability conditions | T 15 days |
| RT | 98.8% |
| +40° C. | 97.6% |

Stability of the clobetasol 17-propionate:

Assay of the active ingredient by internal calibration using HPLC.

The results are expressed in % recovery relative to the theoretical value.

| | Time |
|---|---|
| Stability conditions | T 15 days |
| RT | 98.4% |
| +40° C. | 98.0% |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A sprayable, anhydrous and physically/chemically stable dermatological/pharmaceutical composition, comprising:
a) a therapeutically effective amount of a solubilized corticoid;
b) a therapeutically effective amount of a solubilized vitamin D derivative;
c) an alcohol phase; and
d) an oily phase which comprises one or more oils; formulated into e), a sprayable and topically applicable, dermatologically/pharmaceutically acceptable vehicle therefor.

2. The sprayable, anhydrous dermatological/pharmaceutical composition as defined by claim 1, said corticoid comprising clobetasol propionate.

3. The sprayable, anhydrous dermatological/pharmaceutical composition as defined by claim 2, said vitamin D derivative comprising calcitriol.

4. The sprayable, anhydrous dermatological/pharmaceutical composition as defined by claim 3, said alcohol phase comprising ethanol.

5. The sprayable, anhydrous dermatological/pharmaceutical composition as defined by claim 4, said oily phase comprising one or more oils selected from the group consisting of caprylic/capric triglycerides, cetearyl isononanoate and vegetable oils.

6. The sprayable, anhydrous dermatological/pharmaceutical composition as defined by claim 5, comprising:
a) from 0.0001 to 0.1% of clobetasol propionate;
b) from 0.00001 to 0.1% of calcitriol;
c) from 30 to 60% of ethanol, and
d) from 5 to 80% of an oily phase which comprises one or more oils selected from the group consisting of caprylic/capric triglycerides, cetearyl isononanoate and vegetable oils.

7. The sprayable, anhydrous dermatological/pharmaceutical composition as defined by claim 6, comprising:
a) from 0.001 to 0.05% of clobetasol propionate;
b) from 0.0002 to 0.0005% of calcitriol;
c) from 45 to 55% of ethanol; and
d) from 95 to 99% of an oily phase which comprises one or more oils selected from the group consisting of caprylic/capric triglycerides, cetearyl isononanoate and vegetable oils.

8. The sprayable, anhydrous dermatological/pharmaceutical composition as defined by claim 1, further comprising an antioxidant.

9. The sprayable, anhydrous dermatological/pharmaceutical composition as defined by claim 8, said antioxidant being selected from the group consisting of DL-α-tocopherol, butylated hydroxyanisole and butylated hydroxytoluene.

10. The sprayable, anhydrous dermatological/pharmaceutical composition as defined by claim 1, further comprising a surfactant.

11. The sprayable, anhydrous dermatological/pharmaceutical composition as defined by claim 10, said surfactant being selected from the group consisting of sodium lauryl sulfate, poloxamers and polysorbates.

12. A regime or regimen for preventing or treating dermatological conditions associated with a keratinization disorder relating to differentiation and to proliferation, common acne, comedo-type acne, polymorphic acne, acne rosacea, nodulocystic acne, acne conglobata, senile acne, secondary acne, solar acne, drug-induced acne or occupational acne; ichthyoses, ichthyosiform conditions, Darrier's disease, palmoplantar keratodermas, leukoplakia and leukoplakiform conditions, cutaneous lichen or mucosal (oral) lichen; dermatological conditions having an inflammatory immunoallergic component, with or without a cell proliferation disorder, cutaneous psoriasis, mucosal psoriasis or ungual psoriasis, psoriatic rheumatism, cutaneous atopy, eczema, respiratory atopy or gingival hypertrophy; dermal or epidermal proliferations, benign or malignant, of viral or other origin, common warts, flat warts, verruciform epidermodysplasia, oral or florid papillomatoses and T lymphoma; proliferations induced by ultraviolet radiation, basal cell epithelioma and spinocellular epithelioma; precancerous skin lesions, keratoacanthomas; immune dermatoses, lupus erythematosus; bullous immune diseases; collagen diseases, scleroderma; dermatological or systemic disorders having an immunological component; skin disorders due to exposure to UV radiation, skin aging, light-induced or chronological, or actinic keratoses and pigmentations, or any pathologies associated with chronological aging or actinic aging, xerosis; sebaceous function disorders, hyperseborrhoea of acne, simple seborrhoea or seborrhoic dermatitis; cicatrization disorders or stretchmarks; pigmentation disorders, hyperpigmentation, melasma, hypopigmentation or vitiligo; lipid metabolism ailments disorders, obesity, hyperlipidemia, non-insulin-dependent diabetes or syndrome X; inflammatory disorders, arthritis; cancerous or precancerous conditions; alopecia of various origins, alopecia due to chemotherapy or to radiation; immune system disorders, asthma, type I sugar diabetes, multiple sclerosis, or other selective dysfunctions of the immune system; or disorders of the cardiovascular system, arteriosclerosis or hypertension, comprising spraying onto the affected skin area of an individual in need of such treatment, a thus effective amount of the sprayable, anhydrous dermatological/pharmaceutical composition as defined by claim 1.

13. A regime or regimen for the treatment of psoriasis, comprising spraying onto the affected area of the skin of an individual afflicted with psoriasis, a thus effective amount of the sprayable, anhydrous dermatological/pharmaceutical composition as defined by claim 1.

14. A spray dispenser comprising a housing confining a sprayable, anhydrous dermatological/pharmaceutical composition as defined by claim 1, and a pumping element for mechanically spraying said composition out of said housing.

15. A spray dispenser comprising a housing confining a sprayable, anhydrous dermatological/pharmaceutical composition as defined by claim 1, and a gaseous propellant for spraying said composition out of said housing.

16. The spray dispenser as defined by claim 14, further comprising a metering element for spraying/delivering essentially the same amount of said composition.

17. The spray dispenser as defined by claim 15, comprising an amount of propellant effective for spraying/delivering essentially the same amount of said composition.
